(19) United States
(12) Patent Application Publication
(10) Pub. No.: US 2020/0229973 A1
(43) Pub. Date: Jul. 23, 2020

(54) MOISTURE MIST-SPRAYING DEVICE AND METHOD

(71) Applicant: TSUBOTA LABORATORY, INC., Tokyo (JP)

(72) Inventor: Kazuo TSUBOTA, Tokyo (JP)

(73) Assignee: TSUBOTA LABORATORY, INC., Tokyo (JP)

(21) Appl. No.: 16/697,283

(22) Filed: Nov. 27, 2019

Related U.S. Application Data
(63) Continuation-in-part of application No. PCT/JP2018/021071, filed on May 31, 2018.

(30) Foreign Application Priority Data
May 31, 2017 (JP) 2017-107258

(51) Int. Cl. A61F 9/00; B05B 17/06

(52) U.S. Cl. CPC A61F 9/0026; B05B 17/0676

(57) ABSTRACT

To provide a spraying device and a spraying method for moisture mist that supply at least a micro liquid to the eye, and can be used for dry eye symptom relief, drug delivery, allergy prevention, relaxation, and the like. The above-described problem is solved by a spraying device (1, 31) that supplies at least a micro liquid, and includes a spraying element (16) that sprays a mist-like substance containing the micro liquid onto a local region (50). The raw material is a liquid raw material or a solid raw material that may include a medicinal additive. The medicinal additive is preferably selected from among menthol, analgesics, antibiotics, antiallergic agents, steroids, intraocular pressure-lowering agents, and the like. The spraying device (1, 31) may be an eyeglasses-type spraying device (1) in which the local region (50) is configured by a rim (3) and a temple (6) of eyeglasses, or a cup-type spraying device (31) in which the local region (50) includes an opening (32).

### CROSS REFERENCE TO RELATED APPLICATIONS

This application is a Continuation in Part application of International Application No. PCT/JP2018/021071 filed May 31, 2018, claiming priority based on Japanese Patent Application No. 2017-107258, filed May 31, 2017, the contents of all of which are incorporated herein by reference in their entirety.

### FIELD OF THE INVENTION

The present invention relates to a spraying device and a spraying method for moisture mist that supply at least a micro liquid to the eye. More specifically, the present invention relates to a device and a method for spraying a micro liquid or a micro solid onto a narrow local region near the eye to supply at least the micro liquid to the eye, allowing use for dry eye symptom relief, drug delivery, allergy prevention, relaxation, and the like.

### BACKGROUND ART

Dry eye is a disease in which a stability of a tear film is reduced due to various factors, causing eye discomfort and visual function abnormalities, and may be accompanied by damage to the eye surface. Reportedly, the number of dry eye patients is as many as approximately 22 million in Japan, and continues to increase in a modern society in which a greater number of people use computers and smartphones. For dry eye patients, tears have less volume or are more readily evaporated than healthy persons, resulting in eyes in a dry state. For treatment, eye drops and punctal plugs are normally used, but alternative symptom relief measures are being investigated.

For example, Patent Document 1 proposes eyeglasses for dry eyes capable of controlling the suppression and promotion of moisture transpiration in a well-balanced manner and stably supplying moisture for a long period. These eyeglasses for dry eyes include a moisture transpiration body that transpires moisture on an inner side of a temples of an eyeglasses frame, a heat receiver that comes into contact with a temporal area when the eyeglasses are worn to receive a transfer of heat from the temporal area, and a heat conductor that transfers the heat of the heat receiver to the moisture transpiration body, and thus promotes moisture transpiration by a body temperature of the temporal area when worn.

Further, Patent Document 2 proposes an eyeglasses frame for dry eyes that is excellent in design and has a high moisturizing effect, and eyeglasses for dry eyes including the same. This eyeglasses frame includes a front part configured to be capable of mounting lenses to the left and right, and a pair of left and right temple parts that hook around the ears of a wearer. At least an interior of a lower portion of a rim has a cavity, a plurality of transpiration holes are made in an upper surface of the lower portion of the rim, a moisturizing liquid is injected into the cavity from an injection port on a side portion of the rim, and the moisture of the liquid is transpired from the transpiration holes, thereby humidifying the area around the eye.

Further, Patent Document 3 proposes eyeglasses including a liquid storage part that is arranged in an interior of a frame and forms a cavity for storing liquid. The liquid storage part of the eyeglasses includes a gas permeation part that allows the permeation of gas, but not liquid, from the cavity to a surface on an inner side of the frame opposing the face when the frame is worn on the face, and humidifies the area by vapor pressure in accordance with atmospheric temperature.

It should be noted that, according to a relationship between dry eyes and humidity described in Non-Patent Document 1, it is reported that, while an evaporation rate of tears decreases as humidity rises, a difference exists between dry eye patients and healthy persons at a moderate humidity (40%), both dry eye patients and healthy persons have zero evaporation rates at a high humidity (70%), and thus a high humidity has a positive effect on dry eyes.

### Patent Documents

Patent Document 1: Japanese Laid-Open Patent Application No. 2011-253100
Patent Document 2: Japanese Laid-Open Patent Application No. 2012-73615
Patent Document 3: Japanese Laid-Open Patent Application No. 2012-137694

### Non-Patent Documents

Non-Patent Document 1: Louise C. Madden, et. al., Eye & Contact Lens, 39 (2), p. 169-174 (2013)
Non-Patent Document 2: T. Quallo, et. al., Nature Communications, 6: 7150 doi: 10. 1038/ncomms 8150 (2015)

### SUMMARY OF THE INVENTION

### Problems to be Solved by the Invention

In a dry room, eyes also readily become dry, making dry eye symptoms even worse. In order to prevent a room from becoming dry, it is convenient to adjust the humidity using a humidifier or air conditioner, and generally it is said that a humidity of 40% to 60% is optimal. However, according to the above-described Non-Patent Document 1, such a humidity is not sufficient for eyes exhibiting dry eye symptoms. When a humidifying function of the humidifier or the air conditioner is used in an attempt to increase the humidity of the room in order to relieve dry eye symptoms, the humidity of the entire room increases and a significant amount of power is required. Furthermore, furniture, electrical appliances, and the like that are sensitive to humidity are dampened, causing, for example, a television screen to become fogged due to moisture or moisture to soak into an internal electrical circuit or wiring.

Further, the eyeglasses for dry eyes described in Patent Documents 1 to 3 are still not sufficient for suppressing dry eye symptoms.

An object of the present invention is to provide a spraying device and a spraying method for moisture mist that supply at least a micro liquid to the eye, and can be used for dry eye symptom relief, drug delivery, allergy prevention, relaxation, and the like.

### Means for Solving the Problems

The present inventor has been investigating means for spraying vapor (also referred to as moisture) onto a local region around the eye as means for effectively relieving dry eye symptoms. During the investigation process, it was discovered that spraying a mist-like substance (also referred to as mist) of a micro liquid together with or separately from the vapor can increase an environmental humidity in and moisturize a local region, cause the micro liquid to reach the eye, and increase a tear volume. As a result of further investigation based on such means, it was also discovered that causing a medicinal additive or the like to be contained in a micro liquid is effective as a drug delivery technique for the eye. The present invention was made on the basis of these findings. It should be noted that, in the present application, “moisture mist” refers to a mist-like substance containing at least a micro liquid.

(1) A spraying device according to the present invention is a device that supplies at least a micro liquid, and comprises a spraying element that sprays a mist-like substance containing the micro liquid onto a local region.

According to this invention, the spraying element that sprays a mist-like substance containing a micro liquid onto a local region is included and thus, by mounting this device (specifically, a spraying direction from the spraying element) oriented toward the eye, it is possible to form a local region around the eye and spray a mist-like substance containing a micro liquid onto the local region. A portion of the sprayed mist-like substance containing the micro liquid is vaporized in the local region and becomes a gas (a vapor, for example), but the remaining mist-like substance containing the micro liquid is supplied to the eye without becoming a gas. As a result, the mist-like substance containing the micro liquid can reach the eye in a state of increased gas concentration in the local region, and the device can be used for various applications (dry eye symptom relief, drug delivery, allergy prevention, relaxation, and the like).

In the spraying device according to the present invention, the spraying device further comprises a storing part for storing a raw material of a mist-like substance containing the micro liquid. According to this invention, the raw material stored in the storing part is sprayed as a mist-like substance containing the micro liquid by the spraying element.

In the spraying device according to the present invention, the storing part may be a detachable cartridge container. According to this invention, the cartridge container storing the raw material of the mist-like substance containing the micro liquid need only be replaced, and thus a convenient form of use can be achieved.

In the spraying device according to the present invention, preferably the raw material is a liquid raw material or a solid raw material that may include a medicinal additive. According to this invention, with the raw material containing a medicinal additive, the spraying device can have a drug delivery function of supplying a medicinal substance to the eye. The medicinal additive is preferably a substance for spray treatment, and examples include menthol, analgesics, antibiotics, antiallergic agents, steroids, intraocular pressure-lowering agents, and the like. For example, when menthol is contained as a medicinal additive, menthol can be blown into the eye, and the cold stimulus receptors TRPM8, present in the cornea, are activated, providing a cool sensation while conversely warming the body. According to Non-Patent Document 2, it is suggested that TRPM8 may be related to blinking as well, and spraying menthol promotes blinking and, as a result, can reduce dry eye symptoms.

In the spraying device according to the present invention, preferably the raw material contains physiological saline. According to this invention, the raw material containing physiological saline is sprayed as a micro liquid and supplied to the eye, and thus can be supplied without the eye stinging.

In the spraying device according to the present invention, the spraying element includes an ultrasonic oscillation element and/or an atomizing element. According to this invention, the ultrasonic oscillation element can vibrate the raw material to produce at least a micro liquid, and the atomizing element can produce a reduced pressure state based on air flow to turn a liquid into a mist-like micro liquid. It should be noted that the spraying element may simultaneously be used as an electric resistance element capable of heating the raw material.

In the spraying device according to the present invention, the spraying device may further comprise a heating element that heats the mist-like substance containing at least the micro liquid sprayed by the spraying element. According to this invention, the mist-like substance sprayed by the spraying element can be heated, making it possible to control the mist at a proper sensitive temperature.

In the spraying device according to the present invention, preferably the spraying device is an eyeglasses type with the local region configured by rims and temples of eyeglasses. In this case, preferably the spraying element is provided to the temples.

In the spraying device according to the present invention, preferably the spraying device is a cup type with the local region including an opening. According to this invention, the mist-like substance containing the micro liquid can be sprayed from the opening of the cup toward the eye.

(2) A spraying method according to the present invention comprises a step of using the above-described spraying device according to the present invention to spray at least a micro liquid onto the eye.

### Effect of the Invention

According to the present invention, a local region is formed around the eye and a mist-like substance containing a micro liquid can be sprayed onto the local region, and thus a portion of the sprayed micro liquid is vaporized in the local region and becomes a gas (a vapor, for example), but the remaining micro liquid is supplied to the eye without becoming a gas. As a result, the micro liquid can reach the eye in a state of increased gas concentration in the local region, and the spraying device and spraying method can be used for various applications (dry eye symptom relief, drug delivery, allergy prevention, relaxation, and the like).

### BRIEF DESCRIPTION OF THE DRAWINGS

FIG. 1 is a schematic view of a micro liquid and a gas sprayed by a spraying device and a spraying method according to the present invention.

FIG. 2 is an explanatory view of a mode realized by the spraying device and the spraying method according to the present invention.

FIG. 3 is a schematic view illustrating an example of an eyeglasses-type (closed-type) spraying device according to the present invention.

FIG. 4 is a schematic view illustrating an example of an eyeglasses-type (opened-type) spraying device according to the present invention.

FIG. 5 is a schematic view of when the eyeglasses-type spraying device illustrated in FIG. 3 is worn.

FIG. 6 is a schematic view illustrating an example of when a storing part is detachable.

FIG. 7 is a schematic view illustrating an example of when a humidity sensor is mounted.

FIG. 8 is a schematic view illustrating an example of a cup-type spraying device according to the present invention.

FIG. 9 is a graph showing a relationship between a humidity in an area between the eyeglasses and the eyes and an activation time of the eyeglasses-type spraying device.

FIG. 10 is a graph showing the relationship between the humidity and the activation time when an ultrasonic oscillation element is subject to ON/OFF control.

FIG. 11 is a graph showing a change in humidity in a space in front of the right eye when water is sprayed at a cycle of ON=30 milliseconds and OFF=100 milliseconds.

FIG. 12 is a graph showing the results obtained in Experiment 3.

FIG. 13 is a graph showing the results obtained in Experiment 4.

FIG. 14 is a graph showing the results obtained in Experiment 5.

FIG. 15 is a graph showing the results obtained in Experiment 9.

FIGS. 16A and 16B is images showing an example in which the spraying device is provided on a brim of a hat.

FIGS. 17A and 17B is graphs showing a change in relative humidity (A) and a change in temperature (B) when the spraying device in 16A and 16B is implemented.

FIGS. 18A and 18B is images showing an example of the eyeglasses-type spraying device.

FIGS. 19A to 19D is an example of an experimental form implemented using the eyeglasses-type spraying device in FIGS. 18A and 18B.

FIG. 20 is a schematic view illustrating a relationship between a spraying schedule, a reached humidity, and an On Time/Period ratio.

FIG. 21 is a graph of when the humidity rises quickly (20 seconds or less) and stably above 90%.

FIG. 22 is an explanatory view of the results of a tear film break-up time (BUT).

### EMBODIMENTS OF THE INVENTION

A spraying device and a spraying method according to the present invention are described below with reference to the drawings. The present invention is not limited to the embodiments and examples below, and various modifications can be made as long as the gist of the present application is included.

### [Spraying Device and Spraying Method]

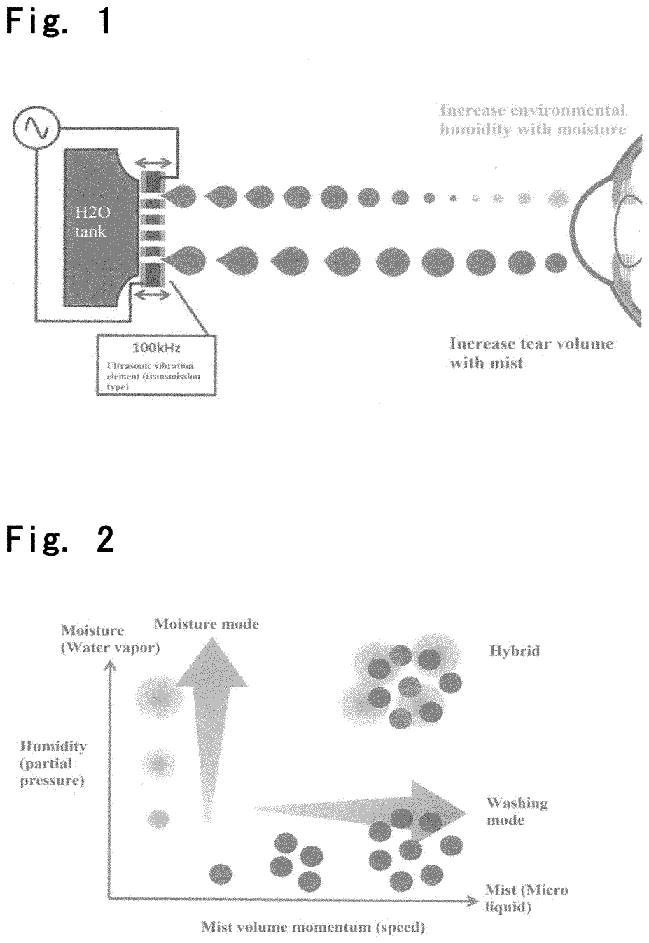

The spraying device and the spraying method according to the present invention are effective means for relieving dry eye symptoms and the like and, as illustrated in FIG. 1, are means for spraying a mist-like substance (mist) containing a micro liquid onto a local region around the eye and supplying at least the micro liquid to the eye. The spray may be only a micro liquid, may be both a micro liquid and vapor (moisture), or may be a micro liquid including a micro solid. Causing a medicinal additive or the like to be contained in a micro liquid is effective as a drug delivery technique for the eye. It should be noted that, in the present application, “moisture mist” refers to a mist-like substance including at least a micro liquid. As illustrated in FIG. 2, the spraying device and spraying method of the present invention can spray a mist-like substance containing a micro liquid (mist), or spray a micro liquid and a gas (moisture) at any ratio. Such spraying makes it possible to exhibit various effects to the eye.

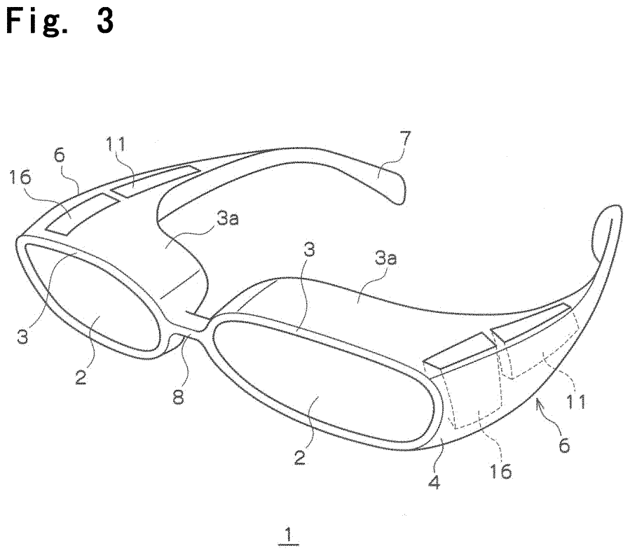

That is, a spraying device (1, 31) according to the present invention, as illustrated in FIG. 3 to FIG. 8, is a device that supplies at least a micro liquid to the eye, and includes a spraying element that sprays a mist-like substance containing the micro liquid onto a local region 50. This device (1, 31) includes the spraying element that sprays a mist-like substance containing a micro liquid onto the local region 50 and thus, by mounting this device oriented toward the eye, it is possible to form the local region 50 around the eye and spray a mist-like substance containing the micro liquid onto the local region 50. The sprayed micro liquid is supplied to the eye, and thus the device can be used for various applications (dry eye symptom relief, drug delivery, allergy prevention, relaxation, and the like). It should be noted that, when a portion of the sprayed micro liquid is vaporized in the local region and becomes a gas (vapor, for example), there is also the advantage that the micro liquid can reach the eye in a moist state of increased gas concentration in the local region 50.

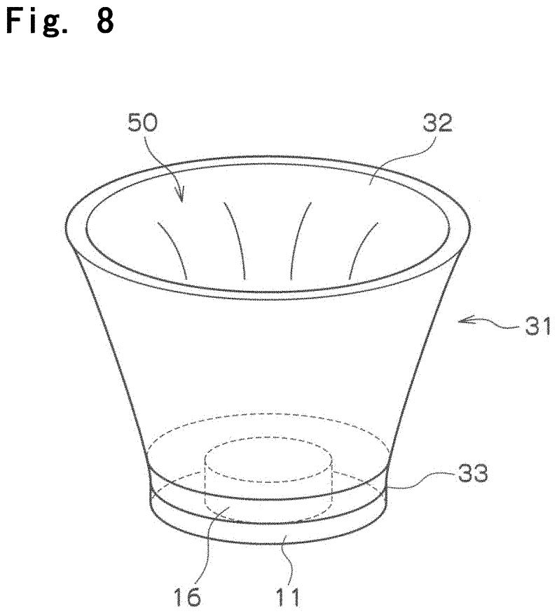

Such a spraying device can be preferably exemplified by an eyeglasses-type spraying device in which the local region 50 is configured by a rim and a temple of the eyeglasses (refer FIG. 3 to FIG. 7), and a cup-type spraying device in which the local region 50 includes an opening (refer to FIG. 8).

Hereinafter, each component of the spraying device will be described in detail, classifying the spraying device into the eyeglasses type and the cup type.

### <Eyeglasses-Type Spraying Device>

As illustrated in FIG. 3 to FIG. 7, the eyeglasses-type spraying device 1 is a device in which a storing part 11 and a spraying element 16 are provided to eyeglasses.

### (Eyeglasses)

A basic structure of eyeglasses, as illustrated in FIG. 3, is generally configured by a lens 2, a rim 3, a wraparound end piece 4, a temple 6, a modern (temple tip) 7, and a nose pad 8. It should be noted that, while normally a hinge is present in eyeglasses, when a projecting section 3a is large as illustrated in FIG. 3, it is difficult to fold the temple 6 with a hinge. Therefore, the eyeglasses need not be provided with a hinge, as illustrated in FIG. 3.

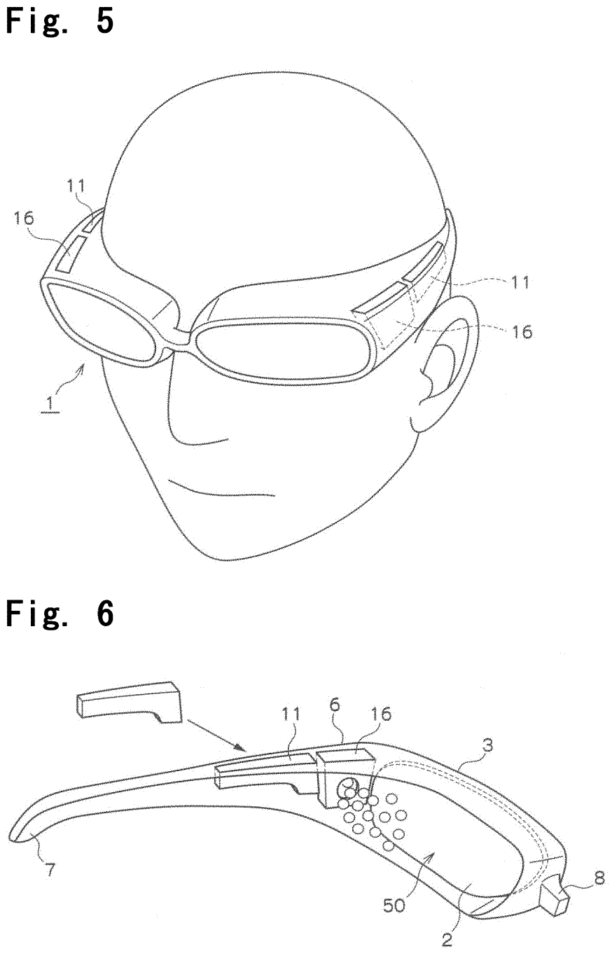

In the eyeglasses-type spraying device 1 according to the present invention, a mist-like substance containing a micro liquid is sprayed onto the local region 50, making it possible for at least the micro liquid to reach the eye. The local region 50 refers to an enclosed space between the eyeglasses and the eye and therefore, as illustrated in FIG. 5, the eyeglasses structure is preferably a closed type in which a gap is not likely to occur between the eyeglasses and the face. As such a structural form, as illustrated in FIG. 3, preferably the projecting section 3a is provided projecting from the rim 3 in a face direction, or a section between the wraparound end piece 4 and the temple 6 is projected in the face direction, making it possible to cover the face and decrease the likelihood of the occurrence of a gap as a result. Further, the temple 6 on the side close to the wraparound end piece 4 may be enlarged, making it possible to decrease the likelihood of the occurrence of a gap between the eyeglasses and the face as a result. When the temple 6 is enlarged to reduce the gap between the eyeglasses and the face, the spraying element 16 and the storing part 11 can be mounted to the temple 6, and thus it is convenient.

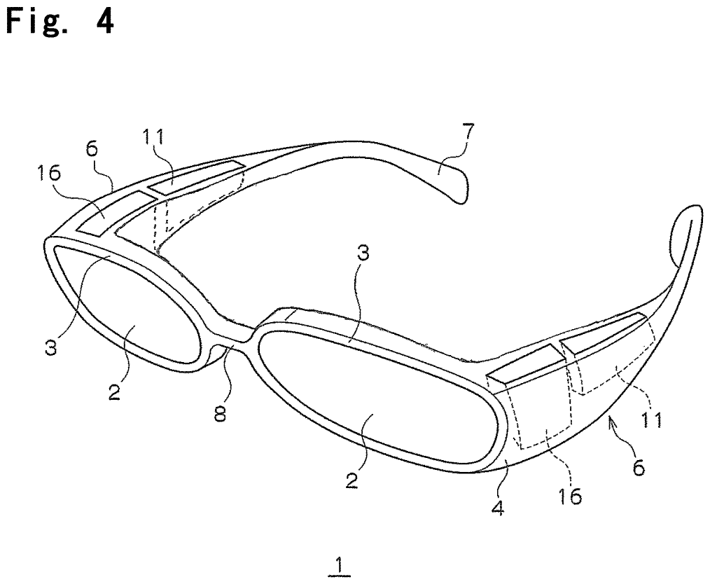

The eyeglasses-type spraying device according to the present invention may have an opened-type eyeglasses structure as illustrated in FIG. 4. Compared to the closed-type eyeglasses structure illustrated in FIG. 3, the opened-type eyeglasses structure is not provided with the projecting section 3a, and further the section between the wraparound end piece 4 and the temple 6 is not projecting in the face direction. Even with such an opened-type eyeglasses structure, the eyeglasses-type spraying device according to the present invention can spray a mist-like substance from the spraying element 16 toward the eye. Therefore, at least the micro liquid can be supplied to the eye.

As described above, in the eyeglasses-type spraying device configured by the rim 3 and the temple 6, preferably the storing part 11 and the spraying element 16 are provided to the temple 6.

The lens 2 may be a glass lens or a plastic lens, and may be a lens capable of cutting a desired wavelength acquired from a lens manufacturer or a glass manufacturer. The materials of the lens 2, the rim 3, the wraparound end piece 4, the hinge, the temple 6, the modern 7, and the nose pad 8 are also not particularly limited, but preferred examples include plastics with good processability, which may be transparent (including colorless and transparent or colored and transparent) or colored and opaque.

### (Spraying Element)

The spraying element 16 is an element that turns the raw material stored in the storing part 11 described later into a mist-like substance and sprays the mist. “Mist-like substance (moisture mist)” refers to a mist containing at least a micro liquid. Preferable examples of the spraying element 16 include an ultrasonic oscillation element capable of vibrating the raw material to produce at least a micro liquid, an atomizing element capable of producing a reduced pressure state based on air flow to turn a liquid into a mist-like micro liquid, and the like. It should be noted that the spraying element may simultaneously be used as an electric resistance element capable of heating the raw material. Regardless of whether a solid or a liquid, the raw material may be sprayed by these spraying elements 16 in a mist form as a micro liquid or a micro solid and ultimately turn into at least a micro liquid when reaching the eye. Accordingly, while not limited to becoming a micro liquid when sprayed, the material becomes a micro liquid when reaching the eye.

The structural form of the spraying element 16 is not particularly limited, but in an example in which the spraying element 16 is provided to the temple 6 as illustrated in FIG. 3 to FIG. 7, is preferably a size and a shape that allow mounting to the temple 6. It should be noted that, when mounted to an area other than the temple 6 as well, preferably the spraying element 16 is made into a size and a shape corresponding to the mounting location.

As the ultrasonic oscillation element, various elements capable of producing a micro liquid (mist) of about several μm (about 1 μm to 10 μm, for example) from a raw material (a solid raw material or a liquid raw material, for example) by ultrasonic atomizing separation can be applied. An operating voltage of the ultrasonic oscillation element is not particularly limited, but the ultrasonic oscillation element is preferably operable with a battery voltage of about 5 V. It should be noted that, as described above, regardless of whether the raw material is a solid or a liquid, this ultrasonic oscillation element may be an element that produces the raw material as a micro liquid or a micro solid, and ultimately turns the raw material into at least a micro liquid when reaching the eye.

The atomizing element is an element that uses an air flow of air or the like and a structural principle of setting a supply path of a liquid to a reduced pressure state and thus spraying a mist-like micro liquid from an air flow path according to the reduced pressure state. The spraying element 16 may include such an atomizing element. Further, the spraying element 16 may be a composite of an ultrasonic oscillation element and an atomizing element.

When the spraying element 16 is an ultrasonic oscillation element 17, the operation control of the ultrasonic oscillation element 17 can be set as desired according to purpose. For example, an intermittent operation (ON/OFF operation or strong/weak operation) may be performed, an output pattern may be set to a pulse, or the mode can be adjusted to “Washing mode,” “Hybrid,” or “Moisture mode” such as illustrated in FIG. 2. As described in an experimental example described later as well, by making the ultrasonic oscillation element 17 of the spraying element 16 operate intermittently, it is also possible to control and regulate the supply of the micro liquid and the gas (water vapor) to ensure that the eyeglasses do not become foggy.

A device capable of adjusting the mode to the “Washing mode,” “Hybrid,” or “Moisture mode” is a device (1, 31) capable of spraying a mist-like substance containing a micro liquid onto the local region 50 around the eye, and includes the storing part 11 that stores a raw material composed of a liquid raw material or a mixture of the liquid raw material and a solid raw material, and the spraying element 16 that produces the mist-like substance containing the micro liquid from the raw material, and the spraying element 16 includes the ultrasonic oscillation element 17 that vibrates the raw material and produces at least the micro liquid. This device (1, 31) includes a control unit that intermittently controls the ultrasonic oscillation element 17 to adjust to a mode selected from the “Washing mode” that uses a large volume of the micro liquid, the “Moisture mode” that uses a large volume of gas, and the “Hybrid mode” that uses the micro liquid and the gas together, and that sprays the micro liquid and the gas at any ratio. This control unit may include an input part for mode switching. Further, the control unit may use a terminal such as a smartphone as an input part and perform mode switching using the input part.

Such a spraying device allows the mode to be switched to any mode according to the purpose of use of the user. Such a mode switching function has never existed in the related art, and can be used for a plurality of improvements in daily life. For example, as described in “Form of use” later as well, it is possible to supply the mist-like substance containing the micro liquid only to a small space at an individual level, for example, around the eyes and around the face, and provide a local space at the individual level without affecting the surrounding area. In particular, the spraying device can be preferably used for dry eye patients. Further, the spraying device can provide a humidity environment more comfortable for the eyes, and further, as a secondary effect, can also moisturize skin around the eyes and help prevent the occurrence of wrinkles in the eye area as well. Further, various effects can be exhibited by adding menthol or the like as a medicinal additive. Furthermore, the spraying device can be used as a humidity holding medical appliance as well, and can be used while carrying out daily life work. Then, the spraying device and spraying method have the effect of improving break-up time (BUT) and dry eye symptoms. In addition, with a medicinal additive being contained, improvements in allergy symptoms and the effects resulting from drug delivery can also be greatly expected.

Further, as illustrated in FIG. 2, the spraying device and spraying method have the advantage that, by controlling the activation of the spraying element 16, it is possible to adjust the mode as desired to “Washing mode” in which a large volume of micro liquid is used, “Hybrid” in which a micro liquid and a gas are used together, and “Moisture mode” in which a large volume of gas (water vapor or the like) is used. With such ease of control, the possibility of new drug delivery can be expected, increasing the spraying amount of micro liquid allows use as a substitute for eye washing as well, allergens can be prevented from entering the eye by forming a water film, and the area around the eye can also be moisturized by maintaining the humidity around the eye.

It should be noted that an electric resistance element capable of heating the raw material may be provided together with such a spraying element 16. As the electric resistance element, various commercially available elements can be applied, and a power saving type electric resistance element can be used as necessary. Due to the ease of changing a raw material (a liquid, for example) into a gas (vapor) by heating, the electric resistance element may simultaneously be used as another spraying element 16. Further, the electric resistant element may be a steam fan type with a fan or the like built-in, and the heated vapor could be made to flow in a specific direction by the fan.

(Storing part)

The storing part 11 is a section that stores the raw material to be turned into a mist-like substance by the spraying element 16. As long as the storing part 11 is configured to have a function capable of supplying the raw material to the spraying element 16, the structural form is not particularly limited. In the example of FIG. 3, a plastic container is mounted to the temple 6. The container may store a liquid or a solid as is, or may store a liquid absorbing material or a liquid retaining material such as sponge or felt soaked with the liquid. The container structure constituting the storing part 11 may be provided integrally with the eyeglasses, or may be a cartridge container detachable from the eyeglasses as illustrated in FIG. 6. In either case, the storing part 11 may be configured to include a replenishing port for replenishing the raw material, or an opening allowing replacement of the raw material.

As illustrated in FIG. 3 to FIG. 7, the storing part 11 is preferably provided to the temple 6, but may be provided to an area of the eyeglasses other than the temple 6. Further, in some cases, a separate container may be connected to the eyeglasses by a tube or the like. The raw material supplied from this storing part 11 to the spraying element 16 can be sprayed as a mist-like substance from the spraying element 16 onto the local region 50 near both eyes.

The raw material may be a liquid raw material, a solid raw material, or a mixture thereof. The raw material may be a material that sprays as a micro liquid or a micro solid by the spraying element 16, and ultimately at least becomes a micro liquid when reaching the eye. In general, use of a liquid is preferred. Other than a liquid, the raw material may be material that becomes a micro liquid before reaching the eye or, for example, a micro solid may be sprayed by the spraying element 16, or a mixture of a micro liquid and a micro solid may be sprayed.

The liquid raw material is a material conveniently used in the present invention, and is a liquid such as water, an aqueous solution, or an organic solvent, but is not limited thereto. The aqueous solution is water containing a water-soluble substance or an aqueous solvent such as a water-containing ethanol, and examples of the water-soluble substance include inorganic substances or organic substances dissolved or dispersed in water. The water is not particularly limited as long as clean, and may be tap water, commercially available mineral water, or the like, or may be distilled water or ion exchange water. The organic solvent is also not particularly limited, and various organic solvents may be applied as long as the effects of the present invention are not impaired.

Examples of the solid raw material include those that become a micro solid and turn into a mist-like substance by an ultrasonic oscillation element, micro solid particles such as nanoparticles, and the like. Such a micro solid may be a mist-like substance of a solid as is or a mist-like substance nano-dispersed in a micro liquid.

The raw material may contain an additive. The additive may be, for example, a salt constituting physiological saline, or may be a fragrance, a cooling agent, an antiseptic, a bactericide, a potential hydrogen (pH) adjuster, a stabilizer, a medicinal additive, or the like. The content of these additives may be as desired within a range that does not impair the effects of the present invention, used for various applications, and is not particularly limited.

When a medicinal additive is contained as an additive, preferably the raw material is a liquid raw material or a solid raw material including the medicinal additive. With the raw material containing a medicinal additive, the spraying device can have a drug delivery function of supplying a medicinal substance to the eye. The medicinal additive is preferably a substance for spray treatment, and examples include menthol, analgesics, antibiotics, antiallergic agents, steroids, intraocular pressure-lowering agents, and the like. For example, when menthol is contained as a medicinal additive, menthol can be blown into the eye, and the cold stimulus receptors TRPM8, present in the cornea, are activated, providing a cool sensation while conversely warming the body. According to Non-Patent Document 2, it is suggested that TRPM8 may be related to blinking as well, and spraying menthol promotes blinking and, as a result, can reduce dry eye symptoms. Examples of other medicinal additives include pharmacologically active components, physiologically active components, and the like. The types of such components are not particularly limited, and examples thereof include a decongestant component, an ocular muscle regulator component, an antiinflammatory component, an astringent component, an antihistamine component, an antiallergic component, vitamins, amino acids, antibiotic components, saccharides, polymer compounds or derivatives thereof, cellulose or derivatives thereof, local anesthetic components, glaucoma treatment components, cataract treatment components, and the like. With such medicinal additives supplied to the eye together with the micro liquid, the respective medicinal effects can be expected.

The content of the medicinal additive may be within a range that does not impair the effects of the present invention, in accordance with the type of medicinal additive.

### (Heating Element)

The eyeglasses-type spraying device 1 may further include a heating element (not illustrated). This heating element is different from the electric resistance element described above, and is an element for supplying the mist-like substance produced by the spraying element 16 to the eye in a further warmed state. For example, when the raw material becomes a gas or the micro liquid becomes a gas, a temperature reduction occurs by vaporization heat, but the temperature felt in the eye or around the eye can be heated to a proper temperature with this heating element.

### (Other)

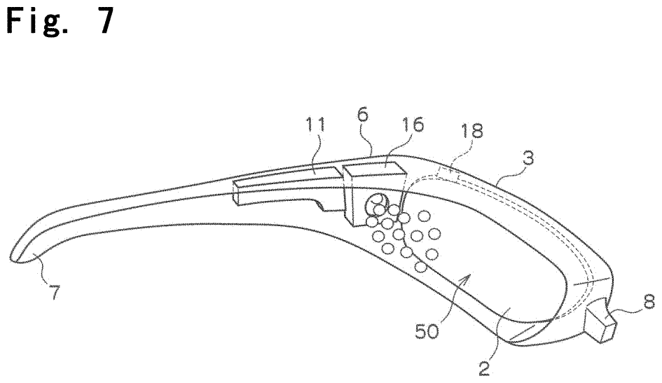

A humidity sensor 18 or a temperature sensor may be provided in the local region 50 (for example, on an inner side of the eyeglasses), as illustrated in FIG. 7. The humidity sensor 18 measures the humidity of the space in which the mist-like substance is sprayed, and can be applied to an operation control of the spraying element 16 based on the measured humidity. Further, the temperature sensor measures the temperature of the space in which the mist-like substance is sprayed, and can be applied to an operation control of the spraying element 16 based on the measured temperature and an operation control of the above-described heating element. It should be noted that the humidity sensor 18 and the temperature sensor may be a temperature and humidity sensor capable of measuring temperature and humidity at the same time.

### <Cup-Type Spraying Device>

The cup-type spraying device 31 can be configured such that at least the spraying element 16 is provided to a cup, as illustrated in FIG. 8. In the cup-type spraying device 31, it is normally preferable that the raw material storing part 11 and the spraying element 16 are provided at a bottom portion 33 of the cup, and an opening 32 of the cup is brought into contact with or close to the eye, making it possible to supply a mist-like substance to the local region 50 in the vicinity of the eye.

The material of the cup is not particularly limited, and plastic is preferably applicable. It should be noted that the structures and elements of the storing part 11 and the spraying element 16 are the same as those described in the description area of the eyeglasses-type spraying device 1, and thus descriptions thereof are omitted here.

In this way, the cup-type spraying device is a device that uses a cup including an opening, and is configured by making a hole in the bottom (bottom portion 33) of the cup to install the spraying element 16, and further install the storing part 11 on a back surface thereof

### <Other Spraying Device>

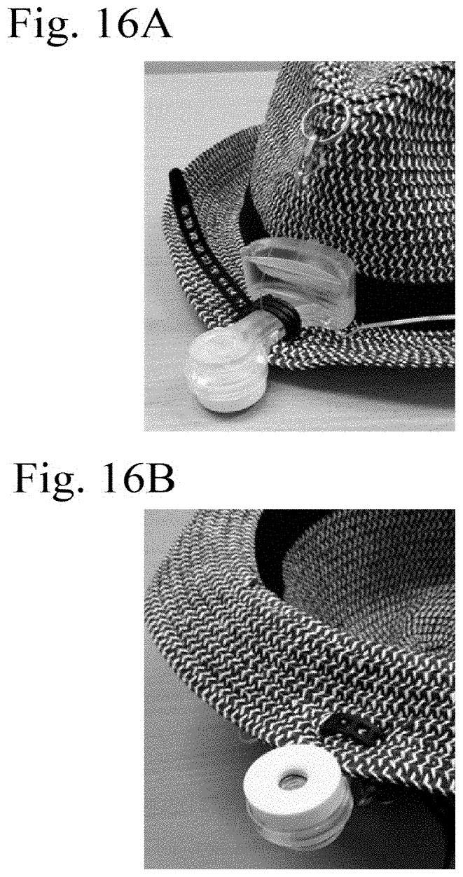

While an eyeglasses-type (closed-type, opened-type) spraying device and a cup-type spraying device have been described as specific embodiments in the above, the spraying device may be another form as long as capable of supplying at least a micro liquid to the eye. For example, as illustrated in FIG. 16 described later, the present invention also includes a configuration in which the spraying element 16 and the storing part 11 are provided on a brim of a hat and a mist-like substance sprayed from the spraying element 16 is directed to the eye. At this time, the spraying element 16 need not be provided on the brim of the hat, and may be attached to the hat via an arm. Further, the spraying element 16 may be provided to a tip end of the arm in the form of a so-called head set. It should be noted that the storing part 11 may be integrally provided near the spraying element 16, but in the case of a hat type or a head set type, the storing part 11 may be connected to the spraying element 16 by a tube or the like, allowing a large size of a storing part 1 to be provided to another area, which is advantageous.

That is, the hat-mounted type spraying device illustrated in FIG. 16 is provided with a spraying element part, including the spraying element 16, and the storing part 11 on the brim of the hat, the spraying element part includes the spraying element 16 that sprays mist from an opening at a center portion of a circular member so that the mist-like substance is directed to the eye, and the storing part 11 is connected and integrally provided to the spraying element part.

### <Form of Use>

The spraying device and the spraying method according to the present invention can spray a mist-like substance containing a micro liquid in a spatially narrow region (local region 50), but do not significantly affect other spaces. Therefore, even when the wearer moves around, it is possible to supply a mist-like substance containing a micro liquid only to a small space at an individual level, for example, around the eyes and around the face, and provide a local space at the individual level without affecting the surrounding area.

A representative example of the main target of this invention is a dry eye patient, but is not particularly limited. For example, for a dry eye patient, at least a micro liquid is sent into the local region 50 in the vicinity of the eyes, and thus a humidity environment more comfortable for the eyes can be provided. Further, as a secondary effect, the spraying device and the spraying method can also moisturize skin around the eyes and help prevent the occurrence of wrinkles in eye area as well. Furthermore, various effects can be exhibited by adding menthol or the like as a medicinal additive.

Further, the spraying device and the spraying method according to the present invention can be used as, for example, a humidity holding medical appliance as well, can be used while carrying out daily life work, and further can suppress the fogging of eyeglasses as well by controlling the spraying element 16 as confirmed in the experimental example described later. Furthermore, it is also possible to increase the humidity of the local region 50 as desired during wearing, improve dry eyes and the like during wearing, and stop wearing at any time. In particular, this spraying device and spraying method are effective immediately due to the action of the spraying element, bring about an awareness in the patient that a difference can be felt, easily control environmental humidity, and further are highly safe. Then, the spraying device and spraying method have the effect of improving break-up time (BUT) and dry eye symptoms. In addition, with a medicinal additive being contained, improvements in allergy symptoms and the effects resulting from drug delivery can also be greatly expected.

Further, as illustrated in FIG. 2, the spraying device and spraying method have the advantage that, by controlling the activation of the spraying element 16, it is possible to adjust the mode as desired to “Washing mode” in which a large volume of micro liquid is used, “Hybrid” in which a micro liquid and a gas are used together, and “Moisture mode” in which a large volume of gas (water vapor or the like) is used. With such ease of control, the possibility of new drug delivery can be expected, increasing the spraying amount of micro liquid allows use as a substitute for eye washing as well, allergens can be prevented from entering the eye by forming a water film, and the area around the eye can also be moisturized by maintaining the humidity around the eye.

### Examples

The present invention is described in further detail below using examples. It should be noted that the subjects in each experiment only refer to the numbers of the subjects in the experiment, and “subject 1” and “subject 2” do not mean the same persons in all experiments.

### Experiment 1

The eyeglasses-type spraying device (Device No. 1) illustrated in FIG. 3 was fabricated. As the eyeglasses frame, JINS MOISTURE (product number: MST-13A-003, JINS Inc.) was used, and the storing part 11 and the spraying element 16 were mounted in a space formed by removing a water tank of the temple 6 on the left side. A plastic container (capacity: approximately 5 mL) housing a sponge soaked with water was used as the storing part 11, and an area of the sponge in contact with the spraying element 16 was adjusted so that a proper amount was in contact with the spraying element 16. As the spraying element 16, a liquid-permeating ultrasonic vibration plate (oscillation frequency 120 kHz, power supply 5 V/500 mA) was used. This ultrasonic oscillation plate includes micropores, and water in contact with one plane can be passed through the micropores and turned into a mist. With this Device No. 1, the produced mist was sent into the space between the eyeglasses and the eye.

### (Humidity Measurement 1)

FIG. 9 is a graph showing a relationship between a humidity in an area between the eyeglasses and the eyes and an activation time of Device No. 1. The hygrometer used was a thermometer and hygrometer evaluation kit (EK-H4) manufactured by Sensiron, the temperature and humidity sensor used was also SHT-21 manufactured by the same company, and these were attached to the inner side of the eyeglasses to acquire data. As illustrated in FIG. 9, Device No. 1 was activated for 20 seconds, causing a relative humidity of 47% to rise to 93% in the eyeglasses and become saturated. After operation for a total of 30 seconds, the ultrasonic oscillation element was turned off, but the humidity saturation subsequently continued for a while. As moisture escaped from the gap between the eyeglasses and the face as time elapsed, it was observed that the humidity gradually decreased and returned to the original environmental humidity over a period of five to six minutes. The measurement results of changes in humidity over time are described below.

### (Humidity Measurement 2)

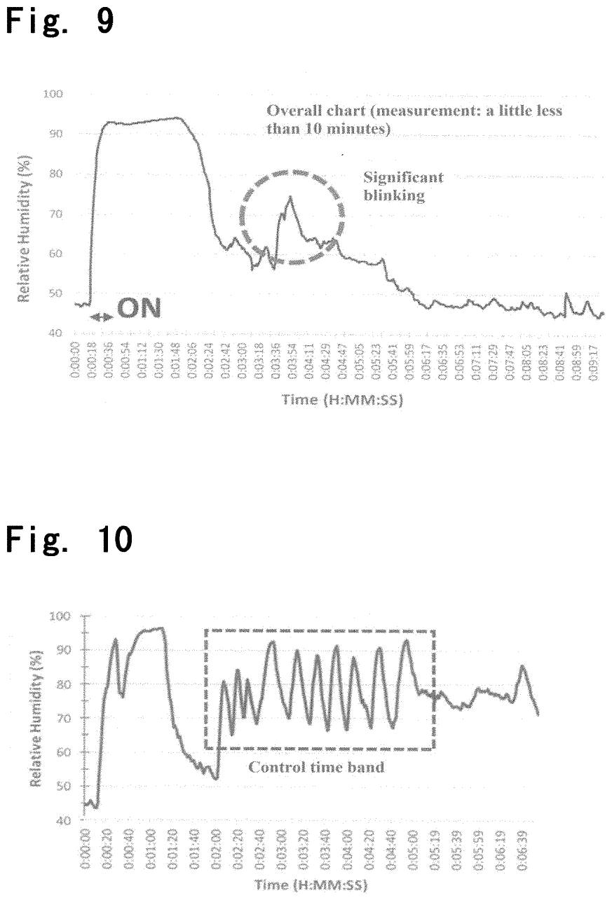

FIG. 10 is a graph showing the relationship between humidity and activation time when the ultrasonic oscillation element was subject to ON/OFF control. As shown in FIG. 10, the humidity could be kept within a range of 80%±10%.

### Experiment 2

The eyeglasses-type spraying device (Device No. 2) was fabricated. As this Device No. 2, a liquid-permeating ultrasonic oscillation plate (vibration frequency: 100 kHz, power supply: 5 VDC, micro-USB connecting) was used for the spraying element 16, a container having a capacity of approximately 5 mL was used for the storing part 11, and this container and ultrasonic oscillation plate were mounted to the temples 6 on the left and right in a sprayable manner. The ultrasonic oscillation plate was activated so as to alternately turn ON and OFF on the left and right with the ON/OFF time electrically set. All other conditions were the same as those of Device No. 1.

### (Humidity Measurement)

FIG. 11 is a graph showing a change in humidity in a space in front of the right eye when water is sprayed at a cycle of ON=30 milliseconds and OFF=100 milliseconds. Compared with Experiment 1, while the oscillation of the ultrasonic oscillation plate was slightly weak and mist tended to escape from the gap around the frame from the start, by slightly sealing the gap at about 4 minutes and 30 seconds, the humidity became approximately 70% and could also be stabilized, as indicated by the arrow in FIG. 11.

### Experiment 3

Using Device No. 1 used in Experiment 1, water mist was jetted into the space in front of the eyes, and a tear film break-up time (BUT) serving as a before-and-after index of dry eye was evaluated. In the experiment, the ultrasonic oscillation plate was activated to increase the humidity to a level exceeding 90%, that state was maintained for 30 seconds, and the before-and-after BUT values were measured. It should be noted that, in Device No. 1, the container and the ultrasonic oscillation plate were only provided to the left eye, and thus only the left eye was measured. The BUT values were obtained by applying approximately 1 μL of fluorescein stain (0.5%) to the eye and measuring the time until the tear film was broken up. Ten seconds or more is normal, and five seconds or less is abnormal.

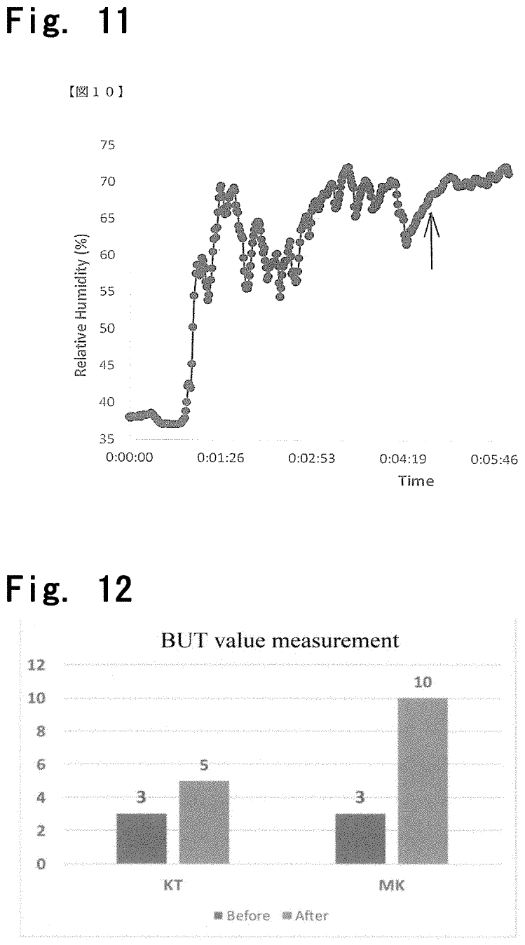

Evaluation was conducted using two subjects. Subject 1 had BUT values of 3 seconds to 5 seconds, subject 2 had BUT values of 3 seconds to 10 seconds, and both showed an increase in BUT value and relief in dry eye symptoms. In particular, the BUT value of subject 2 rose significantly to a normal value of 10 seconds. FIG. 12 is a graph summarizing the results.

### Experiment 4

Using the same Device No. 2 as in Experiment 2, water mist was jetted into the space in front of the eyes, and the BUT values serving as a before-and-after index of dry eye was evaluated. In the experiment, the frame periphery was covered with tape to prevent moisture from escaping, and then the ultrasonic oscillation plate was activated for five minutes so as to alternately turn ON and OFF on the left and right with a repeating cycle of 30 ms ON and 100 ms OFF. The before-and-after BUT values were measured. BUT value measurement was conducted using the same method as that in the above-described Experiment 3. Evaluation was conducted by sensory evaluations of eye comfort each minute, from before starting the experiment to experiment completion. The sensory evaluation had five levels: 0 (zero) at the start, 1 (slightly comfortable), 2 (somewhat comfortable), 3 (comfortable), 4 (quite comfortable), and 5 (very comfortable).

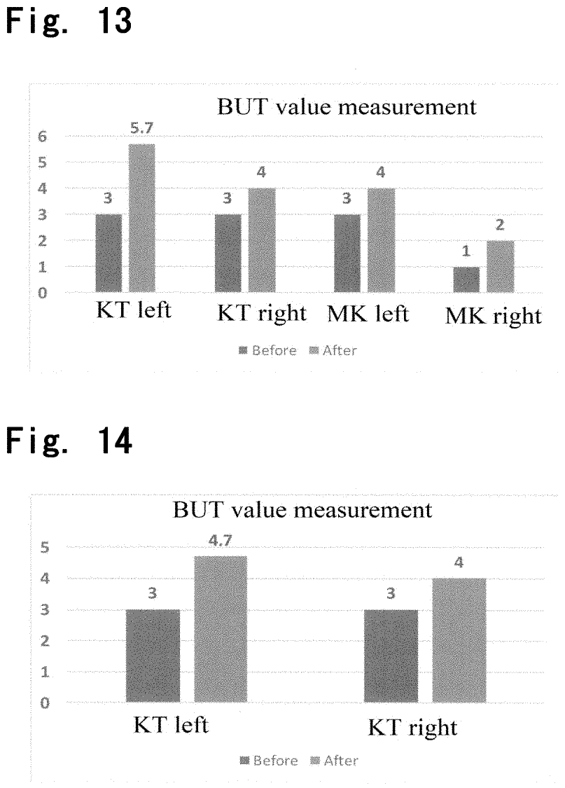

Evaluation was conducted using two subjects. Subject 1 had “left/right” BUT values of 3 seconds/3 seconds to 5.7 seconds/4 seconds, and the sensory evaluation also changed from 0 at the start to 4. Subject 2 also had “left/right” BUT values of 3 seconds/1 second to 4 seconds/2 seconds, and the sensory evaluation also changed from 0 at the start to ¼. FIG. 13 is a graph summarizing the results. Subject 1 showed improvement in BUT values and the sensory evaluation. On the other hand, for subject 2, the sensory evaluation of the left eye with severe dry eye symptoms showed only a slight improvement from 0 to 1, but this is because the power level of the water mist jetting by Device No. 2 was weak, and this can be solved by improving the degree of the spray.

### Experiment 5

Using the same Device No. 2 as in Experiment 2, the ON time was set longer than that in Experiment 2 and the volume of mist jetted per application was increased. Water mist was jetted into the space in front of the eyes, and the BUT values serving as a before-and-after index of dry eye were evaluated. In the experiment, the frame periphery was covered with tape to prevent moisture from escaping, and then the ultrasonic oscillation plate was activated for 15 minutes so as to alternately turn ON and OFF on the left and right with a repeating cycle of 1,024 ms ON and 10 ms OFF. The before-and-after BUT values were measured. BUT value measurement was conducted using the same method as that in the above-described Experiment 3. Evaluation was conducted by sensory evaluations of eye comfort each minute, from before starting the experiment to experiment completion. The sensory evaluation had five levels: 0 (zero) at the start, 1 (slightly comfortable), 2 (somewhat comfortable), 3 (comfortable), 4 (quite comfortable), and 5 (very comfortable).

Evaluation was conducted using one subject. Subject 1 had “left/right” BUT values of 3 seconds/3.3 seconds to 4 seconds/4 seconds five minutes later, and 4.7 seconds/4 seconds ten minutes later. FIG. 14 is a graph summarizing the results. The sensory evaluation also changed from 0 at the start to 4 ten minutes later. Subject 1 showed improvement in BUT value and the sensory evaluation. Further, the lenses of the eyeglasses did not become foggy.

### Experiment 6

Using the same Device No. 2 as in Experiment 2, the mist was thereof jetted into the space in front of the eyes using menthol (Kitami Hakka Tsusho Co., Ltd., “Hakka Oil Set”) diluted by a factor of 500 with ultrapure water, and only before-and-after sensory evaluations were conducted. In the experiment, the frame periphery was covered with tape to prevent menthol moisture from escaping, and then the ultrasonic oscillation plate was activated for five minutes so as to alternately turn ON and OFF on the left and right with a repeating cycle of 30 ms ON and 100 ms OFF in the same way as in Experiment 2. Sensory evaluations were conducted for eye comfort each minute, from before starting the experiment to experiment completion. The sensory evaluation had five levels: 0 (zero) at the start, 1 (slightly comfortable), 2 (somewhat comfortable), 3 (comfortable), 4 (quite comfortable), and 5 (very comfortable).

Evaluation was conducted using one subject. While the sensory evaluation changed from 0 at the start to 4 from after one minute to after five minutes, not many tears were produced. Device No. 2, compared to Device No. 1, had a weak spray, and thus a refreshing feeling was present but not at the highest level and not to the extent that tears were produced.

### Experiment 7

The cup-type spraying device 31 was fabricated. As the cup-type spraying device, an “Eyebon (registered trademark)” manufactured by Kobayashi Pharmaceutical Co., Ltd. was purchased and a prototype of the form illustrated in FIG. 8 was produced using the provided eye cup. As the ultrasonic oscillation element, the same 120 kHz type as the ultrasonic oscillation plate used in Experiment 1 was used. A hole was made in the bottom of the provided eye cup, and the ultrasonic oscillation plate was installed in the hole. As illustrated in FIG. 8, the spraying device was configured so that a mist was sprayed inside the cup by aligning a plastic container (with a sponge inside) against the back surface of the ultrasonic oscillation plate.

An undiluted solution of Eyebon (registered trademark) was used and further diluted with ultrapure water by various magnifications, the mists thereof were jetted into the space in front of the eyes, and before-and-after sensory evaluations (Refreshing feeling, Pain, Tears produced?) were conducted. In the experiment, after the opening of the cup was pressed around the eye, the ultrasonic oscillation plate was activated to spray the mist for 30 seconds. A refreshing feeling was relatively evaluated by being rated as −3 (maximum discomfort), −2, −1, 0 (no change), 1, 2, 3 (maximum refreshing feeling), and pain was relatively evaluated by being rated as 0 (none), 1 (slightly painful), 2 (painful), 3 (very painful).

Evaluation was conducted using one subject. The results of the sensory evaluations are shown in Table 1. As shown in Table 1, it was found that, although not as refreshing as that from normal use of the undiluted solution, a refreshing feeling was obtained even with a dilute solution. When the degree of thinning is low, tears were produced along with pain when the mist was sprayed compared to washing using the undiluted solution. From this, it was understood that use of a certain degree of thinning is required.

TABLE 1

| | | | Diluted by a | Diluted by a | Diluted by a | Diluted by a | Diluted by a |
|---|---|---|---|---|---|---|---|
| Evaluation | Undiluted solution | | factor of 2 | factor of 3 | factor of 4 | factor of 5 | factor of 10 |
| item | Normal use | Ultrasonic wave | | | | | |
| Refreshing feeling | 3 | 2 | 1 | 1 | 1 | 1 | 1 |
| Pain | 0 | 2 | 2 | 1 | 0 | 0 | 0 |
| Tears produced? | No | Yes | Yes | Yes | Yes | Yes | Yes |

### Experiment 8

Using the cup-type spraying device in Experiment 7, the dilution magnification of menthol (Kitami Hakka Tsusho Co., Ltd., “Hakka Oil Set”) was changed using ultrapure water, the mist thereof was jetted into the space in front of the eyes, and before-and after sensory evaluations (Refreshing feeling, Pain, Tears produced?) were conducted. All other conditions were the same as those in Experiment 7.

Evaluation was conducted using two subjects. The results of the sensory evaluations are shown in Table 2. As shown in Table 2, while menthol has strong stimulating and highly refreshing properties, tears were also produced (tear production tends to be refreshing for persons with dry eye symptoms). In addition, pain occurs when the degree of thinning is low. With dilution by a factor of 10 (the strongest), subject 1 rated the evaluation items as Refreshing feeling (3) but Pain (3). On the other hand, subject 2 rated the evaluation items as Pain (3) and Refreshing feeling (0).

TABLE 2

| | | Diluted by a | Diluted by a | Diluted by a |
|---|---|---|---|---|
| | | factor of 10 | factor of 50 | factor of 500 |
| Subject | Evaluation item | Ultrasonic wave | | |
| 1 | Refreshing feeling | 3 | 3 | 3 |
| | Pain | 3 | 3 | 2 |
| | Tears produced? | Yes | Yes | Yes |
| 2 | Refreshing feeling | 0 | 3 | 3 |
| | Pain | 3 | 1 | 0 |
| | Tears produced? | Yes | Yes | Yes |

### Experiment 9

Using the same Device No. 2 as in Experiment 2, an experiment was conducted in which a power of the ultrasonic oscillation plate was increased by a factor of 4 to increase the water jetting volume. The ultrasonic oscillation plate was activated for five minutes so as to simultaneously turn ON and OFF on the left and right with a repeating cycle of 59 ms ON and 41 ms OFF. The before-and-after BUT values were measured. It should be noted that the BUT values were measured in advance (BEFORE measurement), five minutes after activation with the eyeglasses worn (AFTER measurement), and again five minutes after the eyeglasses were removed (final measurement). The evaluation was conducted by sensory evaluations of eye comfort conducted each minute, from before starting the experiment to experiment completion. The sensory evaluation had five levels: 0 (zero) at the start, 1 (slightly comfortable), 2 (somewhat comfortable), 3 (comfortable), 4 (quite comfortable), and 5 (very comfortable). Evaluation was conducted using four subjects.

Subject 1 was a subject with dry eye symptoms. The “left/right” BUT values were BEFORE measurement=2.3 seconds/3.0 seconds, AFTER measurement=7.0 seconds/7.0 seconds, and final measurement=6.3 seconds/5.0 seconds, and thus the state was good for both the AFTER measurement and the final measurement with the eyeglasses removed. Here, when the BUT value was measured further five minutes later, the values were 3.7 seconds/3.3 seconds and were returning to the original values. The sensory evaluations were 0 (BEFORE measurement), 4 (AFTER measurement), 4 (final measurement), and 3 (further measurement after five minutes).

Subject 2 was a subject without dry eye symptoms. The “left/right” BUT values were BEFORE measurement=5 seconds/8 seconds, AFTER measurement=10 seconds or more/10 seconds or more (measurement was performed until 10 seconds and then stopped since the BUT value for a healthy person is 10 seconds or more), and final measurement=10 seconds or more/10 seconds or more. The sensory evaluations were 0 (BEFORE measurement), 4 (AFTER measurement), and 3 (final measurement). This subject 2 originally was without dry eyes and thus had a sense of “wetness,” but otherwise did not feel that the damage to the cornea was healed.

Subject 3 was a subject with dry eye symptoms. The “left/right” BUT values were BEFORE measurement=4 seconds/5 seconds, AFTER measurement=7 seconds/10 seconds, and final measurement=5.5 seconds/4.0 seconds. The sensory evaluations were 0 (BEFORE measurement) and 3 (AFTER measurement), and a final measurement was not performed.

Subject 4 was a subject with dry eye symptoms. The “left/right” BUT values were BEFORE measurement=10 seconds/7.3 seconds, AFTER measurement=10 seconds or more/10 seconds or more, and final measurement=6.7 seconds/8.3 seconds. The sensory evaluations were 0 (BEFORE measurement), 1 (AFTER measurement), and 4 (final measurement). In the AFTER measurement, makeup entered the eyes, making the eyes sting, but removal of the eyeglasses following the AFTER measurement relieved the sting, resulting in a refreshing feeling.

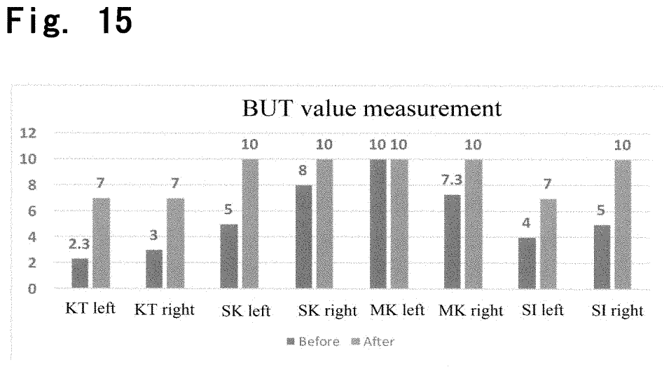

FIG. 15 is a graph showing these results. The BUT average values of the four subjects 1 to 4 were BEFORE measurement=5.6 seconds, and AFTER measurement=8.9 seconds. When a t-test was performed, the p-value was as small as 0.009 and a significant difference was confirmed.

As described above, instead of using a large amount of liquid, a very small amount of liquid was used to fill a local small space with the necessary mist, making it possible to obtain the targeted effect and sufficiently confirm this principle. The experimental results described above clearly show that, by forming a mist with water by an ultrasonic oscillation element and blowing the mist into the local space in front of the eyes, a sense of comfort is felt in the eyes and the BUT value, which is an index of dry eyes, also tends to improve. As for devices, Device No. 1 exhibited enough spray power to exceed a humidity of 90% in a short period, but could only turn ON and OFF, making humidity control difficult. Device No. 2 had the superiority of allowing control of each of the ON/OFF times, but exhibited a relatively weak spray power and increased the humidity only to about 70% (and therefore the eyeglasses did not become foggy). It was also found that moisture leaked from the gap between the eyeglasses frame and the face, and attachment of a cover around the frame to guard against leakage is effective. Further, the enhanced Device No. 2 could increase the spraying volume and was effective.

Further, in view of the possibility of application to drug delivery, by forming a mist with Eyebon and blowing the mist into the eye, it was possible to obtain a refreshing feeling, although dilution was required. To achieve the same washing performance as that when Eyebon (registered trademark) is normally used as an undiluted solution, it is necessary to increase the power of the jetting. When the menthol dilute solution was strong, pain preceded the refreshing feeling, and thus thinning up to a factor of 500 was necessary. When the solution is thinned up to a factor of 500, the pain was also relieved and the refreshing feeling peculiar to menthol could be obtained.

### Experiment 10

FIG. 16 is images showing an embodiment in which the spraying device is provided on a brim of a hat. The spraying device used in this embodiment is provided with a spraying element part and a storing part on the brim of the hat. The spraying element part includes a spraying element that sprays mist from an opening at a center portion of a circular member so that the mist-like substance is directed to the eye. As the spraying element, an ultrasonic oscillation element having a circular shape was used. As this ultrasonic oscillation element, the same liquid-permeating ultrasonic vibration plate (oscillation frequency 120 kHz, power supply 5 V/500 mA) as that in Experiment 1 was used. The storing part is connected and integrally provided to the spraying element part. In the example of FIG. 16, power is supplied to the ultrasonic oscillation element serving as the spraying element from a battery (not illustrated) via a lead wire.

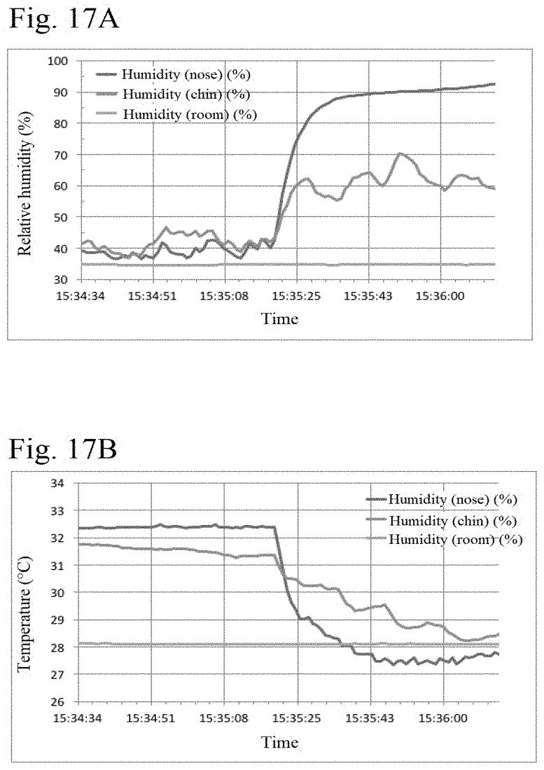

FIG. 17 is graphs showing a change in relative humidity (A) and a change in temperature (B) when the spraying device in FIG. 16 is implemented. As understood from the drawings, when about 40 seconds has passed after the spraying device was turned on, the initial relative humidity and temperature, when measured by the humidity and temperature sensor attached to the nose of the face, changed from about 40% and 32.3° C. to about 90% and 28° C., respectfully, and an increase in relative humidity could be confirmed. Further, the initial relative humidity and temperature, even when measured by the humidity and temperature sensor attached to the chin of the face, changed from about 40% RH and 31.5° C. to about 60% and 28.5° C., respectfully, and an increase in relative humidity could be confirmed. It should be noted that, in all locations, the temperature reduced. The indoor measurement environment was a relative humidity of 35% and an ambient temperature of 28° C.

### Experiment 11

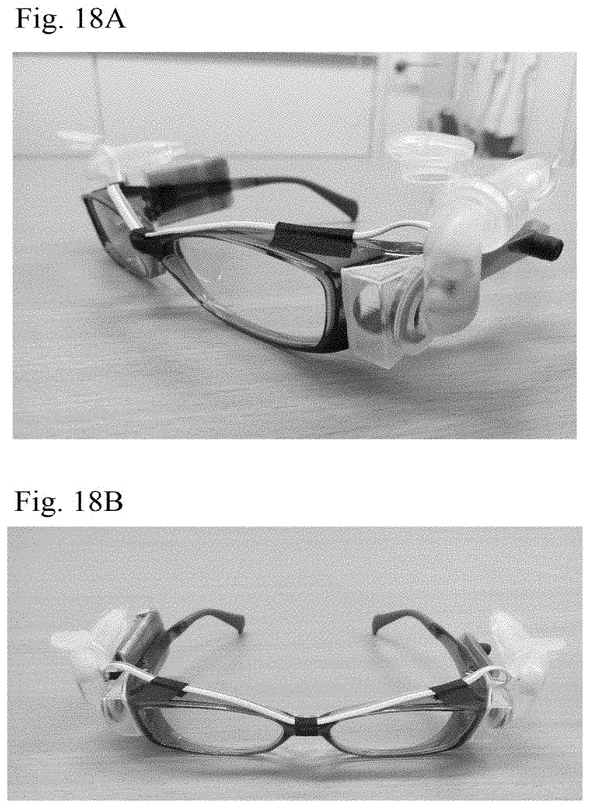

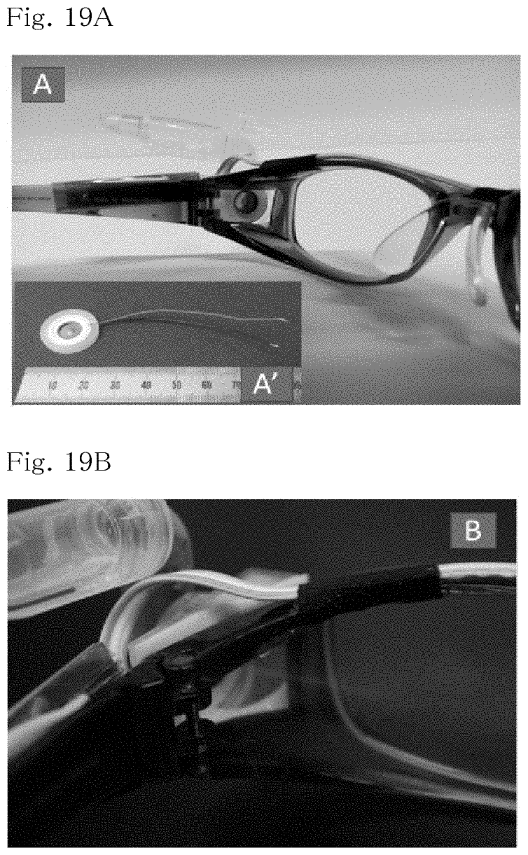

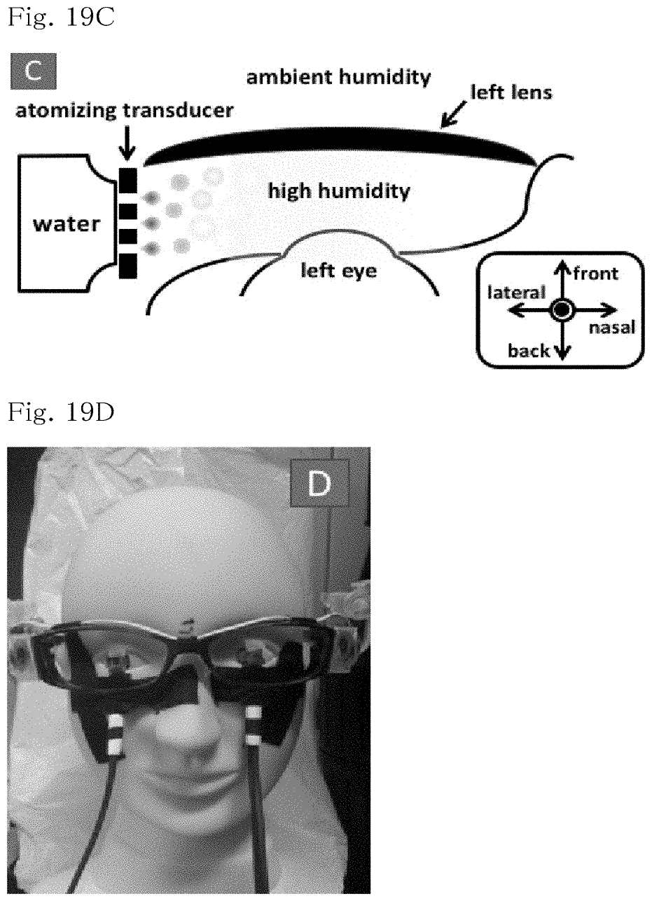

FIG. 18 is images showing an example of an embodiment of the eyeglasses-type spraying device. FIG. 19 is an example of an experimental form implemented using the eyeglasses-type spraying device in FIG. 18. This eyeglasses-type spraying device was a device obtained by further improving Device No. 1 and Device No. 2 used in Experiment 1, and specifically, as illustrated in FIG. 19, the eyeglasses frame used was JINS MOISTURE (product number: MST-13A-003, JINS Inc.), and the storing part and the spraying element were mounted in a space formed by removing a water tank of the temple 6 on the left side. The battery was improved to a chargeable type, from a conventional wired mobile battery or AC power supply connection to a thin lithium polymer battery (power capacity 400 mAh). The storing part was also reduced to a 1.5 mL capacity and changed to a removable cartridge type. Furthermore, use of the sponge used in Device No. 1 and Device No. 2 was also suspended, and a form in which water was introduced directly into the element was adopted. For the electrical system, an input voltage of 5 V from the battery was boosted to ±25 V, and vibration was applied using an ultrasonic piezoelectric transducer (material: lead zirconate titanate) that vibrates at an oscillation frequency of 108 kHz. With such a spraying device, as shown in FIG. 19 (C), a micro liquid can be produced and supplied toward the eye.

It should be noted that the spraying device can be worn on the subject as worn on the mannequin in FIG. 19 (D). In humidity measurement, in the same way as Experiment 1, a thermometer and hygrometer evaluation kit (EK-H4) manufactured by Sensiron was used, a temperature and humidity sensor used was also SHT-21 manufactured by the same company, and these were attached to an inner side of the eyeglasses, making it possible to acquire data.

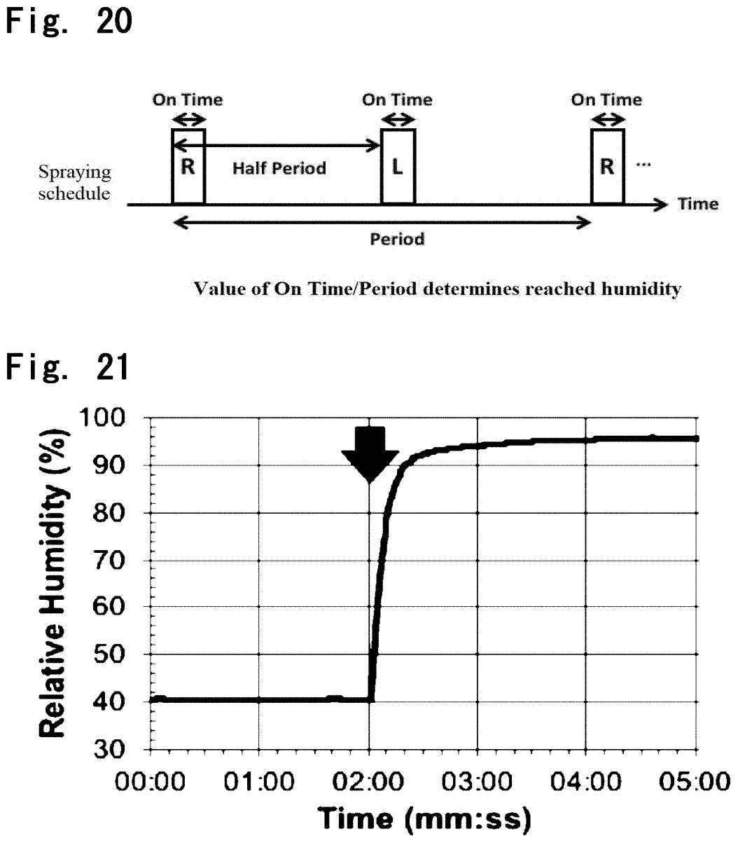

FIG. 20 is a schematic view illustrating a relationship between a spraying schedule, a reached humidity, and an On Time/Period ratio. The ultrasonic vibration element vibrated for each “On Time” and sprayed a micro liquid with a repeating cycle of “Period.” The left and right elements vibrated alternately and were shifted in phase by a half “Period.” From a preliminary experiment using the mannequin shown in FIG. 19(D), it was found that the ratio of “On Time” to “Period” determines the humidity reached in the space in front of the eye.

FIG. 21 is a graph of when the humidity rises quickly (20 seconds or less) and stably above 90%. With the conditions of On Time=8.4 ms and Period=250 ms, On Time/Period=3.4%. From the results in FIG. 21, it was confirmed that the humidity rose rapidly (20 seconds or less) and stably above 90%.

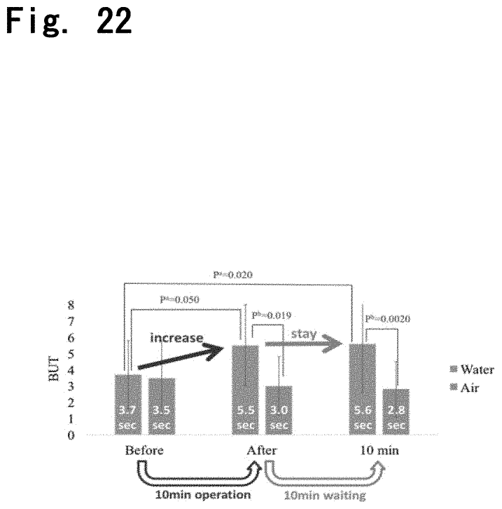

FIG. 22 is an explanatory view of the results of tear film break-up times (BUT). For spraying conditions, the spray strength level was set to On Time/Period=8.4 ms/250 ms=3.4%, the moisture mist was applied for ten minutes, and results and responses of an ophthalmic examination and questionnaire were obtained before and after wearing. The application was conducted twice (two cases: case with water and control (case with air and without water)). The experiment was conducted with five subjects (three men, two women, average age 44.6 years±12.7 years). The main ophthalmic examination items included slit lamp microscopy (tear film break-up time (BUT), keratoconjunctival epithelium disorder level (fluorescein staining score)), tear osmotic pressure test, functional visual acuity (FVA (log MAR scale)), and visual acuity (visual analog scale; VAS). From the results shown in FIG. 22, the tear film break-up time (BUT) value rose significantly after ten minutes of wearing and did not reduce after another ten minutes as well, indicating effectiveness.

Next, the results of functional visual acuity (FVA (log MAR scale)) and blinking frequency per minute are shown in Table 3. From the results in Table 3, both the functional visual acuity (FVA (log MAR)) and the blinking frequency (in one minute) were improved, indicating effectiveness.

TABLE 3

| | Before | After | P Value ^{a} |
|---|---|---|---|
| FVA | | | |
| Water | 0.12 ± 0.16 | 0.05 ± 0.13 | 0.073 |
| Air | 0.12 ± 0.17 | 0.03 ± 0.10 | |
| P Value ^{b} | 0.99 | 0.72 | |
| Blink | | | |
| Water | 19 ± 11 | 13 ± 8 | |
| Air | 21 ± 13 | 20 ± 13 | 0.65 |
| P Value ^{b} | 0.74 | 0.12 | |

Next, the results of visual acuity (visual analog scale; VAS) are shown in Table 4. From the results in Table 4, a superior improvement in visual acuity was seen, indicating effectiveness.

TABLE 4

| | Before | After | 10 min | P Value ^{a} |
|---|---|---|---|---|
| Water | 198 ± 76 | 84 ± 113 | 84 ± 113 | |
| Air | 195 ± 146 | 195 ± 146 | 195 ± 146 | NA |
| P Value ^{b} | 0.97 | 0.22 | 0.22 | |

Next, the fluorescein staining scores are shown in Table 5. From the results in Table 5, there was no change in score, indicating that safety was ensured.

TABLE 5

| | Before | After | P Value ^{a} |
|---|---|---|---|
| Water | 0.40 ± 0.70 | 0.40 ± 0.70 | NA |
| Air | 0.30 ± 0.48 | 0.30 ± 0.48 | NA |
| P Value ^{b} | 0.714 | 0.714 | |

As described above, the spraying device according to the present invention ultrasonically vibrates a piezo element by an inverse piezoelectric effect, and blows the element as water mist (a micro liquid) to humidify the space in front of the eye. Then, because the device includes an electrical control circuit for humidity, a water introduction cartridge (storing part), and the like, the device can be configured to have the same appearance as normal eyeglasses. When a problem occurs in any one of the layers of tears, which have a three-layer structure of oil, water, and mucin, the entire tear film becomes unstable, the keratoconjunctiva are exposed, and the surface is damaged by blinking, and thus the stability of the tear film is essential. In the quantitative index of tear film break-up time (BUT), which measures tear film stability, the BUT value for dry eye patients is short. However, in the spraying device according to the present invention, as shown in each experiment described above, the humidity in the region in front of the eye was increased, the BUT value was lengthened, and a relief in subjective symptoms of dry eye was confirmed.

Furthermore, the spraying device according to the present invention not only relieves eye dryness, but also moisturizes and imparts tension to the skin around the eyes under the same settings, making it possible for an aesthetic effect to be imparted to users concerned about the eye area. In addition, the spraying device can also be used in place of an eyewash, even if the water is blown, by increasing the volume and momentum. When something other than water, for example, a drug in the form of a solution is blown, the spraying device can also achieve a new type of drug delivery.

### DESCRIPTIONS OF REFERENCE NUMERALS

1 Eyeglasses-type spraying device (Eyeglasses)
2 Lens
3 Rim
3a Projecting section
4 Wraparound end piece
6 Temple
7 Modern
8 Nose pad
11 Storing part
16 Spraying element
17 Ultrasonic oscillation element
18 Humidity sensor
31 Cup-type spraying device (Cup)
32 Opening
33 Bottom portion
50 Local region

What is claimed is:

1. A spraying device capable of spraying a mist-like substance containing a micro liquid onto a local region around an eye, comprising:
a storing part that stores a raw material composed of a liquid raw material or a mixture of the liquid raw material and a solid raw material; and
a spraying element that produces a mist-like substance containing the micro liquid from the raw material,
the spraying element including an ultrasonic oscillation element that vibrates the raw material to produce at least the micro liquid.

2. The spraying device according to claim 1, further comprising a control unit that intermittently controls the ultrasonic oscillation element to adjust to a mode selected from a washing mode that uses a large volume of the micro liquid, a moisture mode that uses a large volume of gas, and a hybrid mode that uses the micro liquid and the gas together, and sprays the micro liquid and the gas at an any ratio.

3. The spraying device according to claim 1, wherein
the storing part is a detachable cartridge container.

4. The spraying device according to claim 1, further comprising a temperature sensor, a humidity sensor, or a temperature and humidity sensor.

5. The spraying device according to claim 1, the spraying device being an eyeglasses type, comprising a rim and a temple, wherein
the storing part and the spraying element are provided to the temple.

6. The spraying device according to claim 1, the spraying device being a cup type that uses a cup including an opening, wherein
the spraying element is installed in a hole made in a bottom of the cup, and
the storing part is installed on a back surface thereof.

7. The spraying device according to claim 1, the spraying device being a hat-mounted type provided with a spraying element part, including the spraying element, and the storing part on a brim of a hat, wherein
the spraying element part includes the spraying element that sprays mist from an opening at a center portion of a circular member so that the mist-like substance is directed to the eye, and
the storing part is connected and integrally provided to the spraying element part.

8. The spraying device according to claim 1, wherein
the liquid raw material contains a medicinal additive selected from among menthol, analgesics, antibiotics, antiallergic agents, steroids, intraocular pressure-lowering agents, and the like.

9. The spraying device according to claim 1, further comprising a heating element that heats the mist-like substance containing at least the micro liquid sprayed by the spraying element.

10. A spraying method comprising the step of using the spraying device described in claim 1 to spray at least a micro liquid onto the eye.
